(19) United States
(12) Patent Application Publication
(10) Pub. No.: US 2018/0261330 A1
(43) Pub. Date: Sep. 13, 2018

(54) ANALYTIC AND LEARNING FRAMEWORK FOR QUANTIFYING VALUE IN VALUE BASED CARE

(71) Applicant: ROUNDGLASS LLC, BELLEVUE, WA (US)

(72) Inventor: KANAKA PRASAD SARIPALLI, REDMOND, WA (US)

(21) Appl. No.: 15/917,253

(22) Filed: Mar. 9, 2018

Related U.S. Application Data
(60) Provisional application No. 62/470,150, filed on Mar. 10, 2017.

(51) Int. Cl. G16H 50/30; G06F 15/18; G06F 17/11

(52) U.S. Cl. CPC G16H 50/30; G06F 17/11; G06F 15/18

(57) ABSTRACT

A computer-implemented method includes defining a set of multiple stakeholder entities. For each stakeholder entity of the set of stakeholder entities, a corresponding set of multiple health outcomes (Qᵢ) is defined. A set of multiple costs (Cⱼ) is defined. Each cost of the set of costs corresponds to a respective health outcome (Qᵢ) of the set of health outcomes. For each stakeholder entity of the set of stakeholder entities, a value (V) is determined according to the equation V=(ΣᵢⁿwᵢQᵢ)/(Σⱼ^{m}eⱼCⱼ), wherein wᵢ represents a set of numerical weights and eⱼ represents a set of episodes associated with a particular cost to create a set of values (V). A numerical value (V′) optimal to the set of stakeholder entities is determined from the set of values (V).

### PRIORITY CLAIM

This Application claims the benefit of U.S. Provisional Application No. 62/470,150 filed Mar. 10, 2017, which is hereby incorporated by reference in its entirety as if fully set forth herein.

### BACKGROUND

The current fee-for-service model of healthcare is often criticized as incentivizing healthcare providers, physicians and organizations to target larger volumes—that is, higher numbers of patients, procedures and tests. Value Based Care (VBC), in which providers are paid according to the value they provide to the stakeholder—typically the patient—is gaining momentum as a strategy that will optimize healthcare outcomes. There are no solutions available for the definition, assessment and prediction of Value of Healthcare. According to a National Science Foundation (NSF) panel on VBC, patients view value in healthcare in terms of the quality of their relationship with their physician, helping them better meet their personal goals or living lives that are as normal as possible. It does not necessarily mean more services or more expensive services.

A rigorous definition of value is critical to the success of Value Based Care (VBC), but is not straightforward. This is partly because healthcare treatments address thousands of different disease conditions. Treatments and their outcomes impact many different stakeholders such as, for example, the patient, provider, payer, employer and policy maker. Previous approaches are not based on or amenable to deep (machine) learning approaches, and hence are not able to (i) assess value in detail per each particular treatment among the approximately 16000 defined disease conditions (ii) learn from growing datasets, which is feasible via machine learning (ML) and (iii) predict value even when one or more pertinent inputs are unavailable.

### BRIEF DESCRIPTION OF THE DRAWING FIGURES

FIG. 1 illustrates quality, cost and value calculations for a set of total hip replacement patients according to an embodiment of the present invention;

FIG. 2 illustrates value calculations as a function of patient age for a dataset of a predetermined number of patients according to an embodiment of the present invention;

FIG. 3 illustrates a decision tree for classification of Chronic Obstructive Lung Disease treatments by value according to an embodiment of the present invention; and

FIG. 4 illustrates quality, cost and value calculations for a set of total hip replacement patients according to an embodiment of the present invention.

### DETAILED DESCRIPTION OF THE PREFERRED EMBODIMENT

This patent application is intended to describe one or more embodiments of the present invention. It is to be understood that the use of absolute terms, such as “must,” “will,” and the like, as well as specific quantities, is to be construed as being applicable to one or more of such embodiments, but not necessarily to all such embodiments. As such, embodiments of the invention may omit, or include a modification of, one or more features or functionalities described in the context of such absolute terms.

Embodiments of the present invention may comprise or utilize a special-purpose or general-purpose computer including computer hardware, such as, for example, one or more processors and system memory, as discussed in greater detail below. Embodiments within the scope of the present invention also include physical and other computer-readable media for carrying or storing computer-executable instructions or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the invention can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems or modules or other electronic devices. When information is transferred or provided over a network or another communications connection (either hardwired, wireless, or a combination of hardwired or wireless) to a computer, the computer properly views the connection as a transmission medium. Transmissions media can include a network or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the invention. The computer executable instructions may be, for example, binaries, intermediate format instructions such as assembly language, or even source code.

According to one or more embodiments, the combination of software or computer-executable instructions with a computer-readable medium results in the creation of a machine or apparatus. Similarly, the execution of software or computer-executable instructions by a processing device results in the creation of a machine or apparatus, which may be distinguishable from the processing device, itself, according to an embodiment.

Correspondingly, it is to be understood that a computer-readable medium is transformed by storing software or computer-executable instructions thereon. Likewise, a processing device is transformed in the course of executing software or computer-executable instructions. Additionally, it is to be understood that a first set of data input to a processing device during, or otherwise in association with, the execution of software or computer-executable instructions by the processing device is transformed into a second set of data as a consequence of such execution. This second data set may subsequently be stored, displayed, or otherwise communicated. Such transformation, alluded to in each of the above examples, may be a consequence of, or otherwise involve, the physical alteration of portions of a computer-readable medium. Such transformation, alluded to in each of the above examples, may also be a consequence of, or otherwise involve, the physical alteration of, for example, the states of registers and/or counters associated with a processing device during execution of software or computer-executable instructions by the processing device.

As used herein, a process that is performed “automatically” may mean that the process is performed as a result of machine-executed instructions and does not, other than the establishment of user preferences, require manual effort.

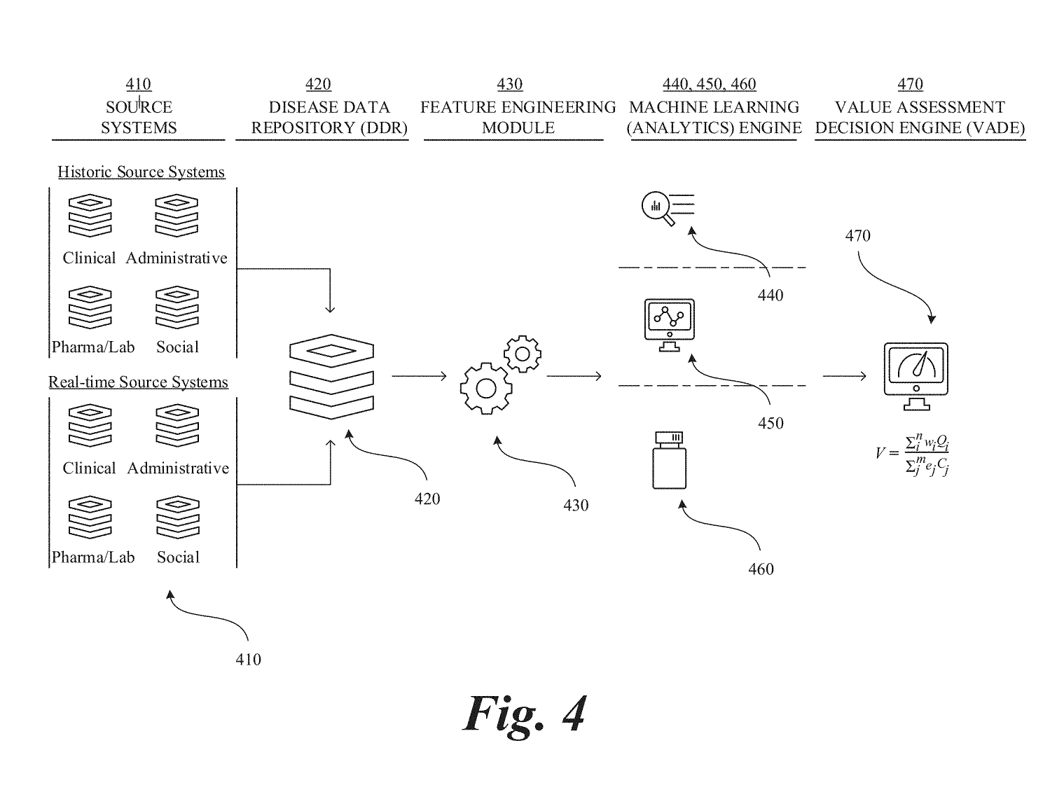

Referring to FIG. 4, and in an embodiment as will be discussed in greater detail herein below, for a given disease or treatment option, relevant data sourcing systems 410 are accessed to ingest required Outcome and Cost data metrics.

A Disease Data Repository (DDR) 420 is constructed for the approximately 16,300 known disease conditions. DDR 420 may include definitions of a set of multiple stakeholder entities, definitions of multiple health outcomes (Qᵢ ) for each stakeholder entity, and definitions of multiple costs (Cⱼ) corresponding to each health outcome (Q′).

A Feature Engineering Module 430 identifes and evaluates the variables (also known as features or attributes) which influence the Outcome Quality (Q), Costs (C) and Value (V) functions based on machine learning (ML) algorithms.

Using ML algorithms known as Recommender Systems, three analytics modules—Descriptive 440, Predictive 450 and Prescriptive 460—rigorously assess and predict V for past and future treatments specific to any of the approximately 16,300 known diseases conditions. The Prescriptive analytics module 460 prescribes one or more preferred treatment options by comparatively assessing the V of such treatments for future treatments.

A Value Assessment Decision Engine 470 determines V according to Equation 1 and a numerical value (V′) specifically to the needs of stakeholder entities.

An embodiment of the invention can be used by healthcare providers, consumers, payers (insurance) and government entities to assess (describe) and/or predict the value of medical and allied procedures and treatments in a rigorous, fully quantitative fashion, and enable the resulting procedure for continuous learning as new data acumulate and become available. One or more embodiments may include an analytics platform based on ML; a mathematical and analytical method; a Software as a Service (SaaS) offering; a Software product; and/or a device for calculation of Value, as defined below. An embodiment includes a fully quantitative analytic and learning framework using ML for assessing Value in VBC as a direct function of the Quality outcomes and Cost metrics, specific to a particular medical treatment. It can be used by various Stakeholders to plan, track, assess and agree upon the Value of a variety of healthcare services, based on rigorous, mathematically tractable computations in a consistent manner. Assessment of Value depends on the stakeholder evaluating it, the specific medical condition and its treatment whose value is being evaluated. We define a stakeholder vector (Payer, Employer, Provider, Consumer (Patient)) or PEPC for this work. The proposed framework via Eq. (1) has the ability to accommodate this diversity, as it is based on integrating Value criteria from all 4 stakeholders.

One or more embodiments include the following features:

Ability to pool, munge, digest and prepare Electronic Health and Medical Records (EHR/EMR) data from a variety of sources to enable the different ML and Image Analyses (IA) engines for a rigorous computation of Value (V).

Specific modules used to collect, survey, analyze and integrate medical, payment and administrative records of Populations, Providers and Payers to serve as the data basis for PEPC Stakeholder matrix.

A deep ML and IA based integrated decision support system each for Value assessment by Module

Automated Hypothesis generation engine to support diagnosis and prognosis

A Recommender System integrated into each of the four PEPCI Value modules to make recommendations for optimizing value while planning, providing, assessing and paying for Care

Several ML components to enable the above including Interestingness Measurement, Clustering, Classification, Prediction, Risk Assessment, Mutli-attribute Decision Making and Recommender Systems.

The proposed method and framework can use the definition of Cost and Outcome Attributes, Alternatives and Attribute Weights, to construct a Decision Matrix and arrive at a relative ranking to identify the optimal alternative. An embodiment also presents a taxonomy organized in a two-level hierarchy based on HEDIS outcome metrics. The Healthcare Effectiveness Data and Information Set (HEDIS) is a performance measurement tool that is coordinated and administered by NCQA (National Committee for Quality Assurance, structured as data collected on 71 measures over 8 domains to compare health plans and hospitals. Methods proposed here with ML algorithms can compute and optimize VBC assessments decisions separately for the four stakeholders (PEPC), where the attributes differently influence the value calculation decisions. A modified Wide-band Delphi method is proposed for assessing the relative weights for each attribute, by Episode. Relative ranks are calculated using these weights, and the Simple Additive Weighting (SAW) method is used to generate value functions for all the alternatives, and rank the alternatives by their value to finally choose the best alternative.

Definition of Value V for each stakeholder (PEPC) for Customer C (Patient), Value of an encounter V can be defined as

$$V = \frac{{\sum }_{i}^{n} {w}_{i} {Q}_{i}}{{\sum }_{j}^{m} {e}_{j} {C}_{j}} \qquad [ Eq . 1 ]$$

where Qᵢ represent the Health outcomes carrying Weights wᵢ, and Cⱼ represent the Costs of delivering the outcomes, while eⱼ is the number of episodes associated with a particular cost contribution Cⱼ. For example, surgery, visitations and pharmacy are three different episodic costs which all contribute to the total Cost. These can be heavily dependent on the specific disease conditions, and hence would be specific to a disease taxonomy described earlier. An embodiment can use the HEDIS metrics a widely used set of performance measures in the managed care industry, as a basis to assess Qᵢ to begin with. An ensemble algorithm optimizes the Value among the four stakeholders to finally arrive at a Value metric which is agreeable to all parties involved. Multi-Attribute Decision making algorithms such as MADM and TOPSIS (Ref) can povide an elaborate matrix for these metrics, mapping to multiple disease and encounter taxonomies, which are established in the EHR practice.

For a Payer, the Value equation would be similar to Eq. 1 above, but the Qᵢ and Cⱼ metrics and weights wⱼ, would be different than those from the Customer's point of view. For example, Costs incurred by a patient and a Payer would be different for the same Encounter. This algorithm would take such differences into consideration. The same reasoning applies to the calculation of Value from a Provider and Employer points of view as well. The desired outcomes, while overlapping, also would be somewhat different for the same Encounter among the four stakeholders PEPC. The proposed methods are fully detailed in the following sections for two different, important disease conditions—Total Hip Replacement (THR) and Chronic Obstructive Pulmonary Disease (COPD), as a part of the detailed description of the invention disclosed here.

One of the advantageous features of the proposed framework is its fully quantitative and iterative convergence approach based on proven multi-attribute decision methods, which enables decision makers to comparatively assess the relative robustness of alternative cloud adoption decisions in a defensible manner. Being amenable to automation, it can respond well to even complex arrays of decision criteria inputs, unlike human decision makers. It can be implemented as a web-based Decision Suppport System (DSS) to readily support cloud decision making world-wide, and improved further using fuzzy TOPSIS methods, to address concerns about preferential inter-dependence of attributes, insufficient input data or judgment expertise.

### Detailed Method Description Applied to THR

A rigorous definition of value is critical to the success of VBC, but is not straightforward. This is partly because healthcare treatments encompass thousands of different disease conditions and treatments, and their outcomes impact many different stakeholders such as the patient, provider, payer, employer and policy maker for example. Even when the product or service being offered is not complex, as in the case of a car, food service etc., consumers perceptions of Price, Quality and Value are difficult to assess. Perceived quality is (1) different from objective or actual quality, (2) a higher level abstraction than a specific attribute of a product or service, (3) a global assessment that in some cases resembles attitude and (4) a judgment usually made within a consumer's evoked set. In contrast, “objective quality” is more closely related to the aggregate of the quality of goods that comprise the product. As it relates to VBC, both Cost and Quality (Q, C) are influenced by perceptions of the stakeholders. For example, even the Cost term, which arguably is an aggregate of all the material costs (dollar value) as per the denominator of Eq. (1), does contain perceived costs which are not necessarily financial and hence not amenable to a dollar cost tag. If a consumer (patient) and their family had to go through considerable hardship getting an appointment for a THR procedure as described later, and getting access to the facilities for accessibility reasons, then they would correctly perceive the additional hardship as a “cost” of the procedure, which would subtract from the overall value of the procedure. Young and Feigen (1975) depict this view in the “Grey benefit chain”, which portrays the perceived benefit of a product or service as the chain: Product→Functional Benefit→Practical Benefit→Emotional Payoff.

Zelthami and others concluded that Value is perceived by consumers in the following 4 ways: (1) value is low price, (2) value is whatever I want in a product, (3) value is the quality I get for the price I pay, and (4) value is what one gets for what one gives. For VBC, this perceived value can be captured via the inclusion of mixed continuous (numerical) and categorical attributes as predictor variables to predict Q, C and hence V, which captures all four definitions above sufficiently. Categorical attributes shown in Table 1 can be designed to adequately capture the three types of benefits (i.e. Outcomes) in the Grey benefit chain (Functional, Practical, and Emotional) as well as the non-material (emotional, etc.) costs. While the Value algorithm (Eq. 1) is compact, its expansion to encompass thousands of disease conditions and stakeholders, at two scales (Individual, Population) makes practical implementation of the proposed framework a very elaborate machine learning project. International Classification of Diseases (ICD) is the standard diagnostic tool for epidemiology, health management and clinical purposes, which provides a complete, detailed taxonomy of more than 14,400 different codes for disease conditions. The predictor attributes and weights entering Eq. (1) for treatment Value calculation or prediction can be specific to each such condition and hence can be uniquely cataloged.

### I. Analytics Framework

### A. Value and Stakeholder Needs

Many studies reported using various econometric approaches to assess cost and benefit of healthcare, but there is as yet no standard practice for measuring value or even an agreed-upon definition of value. Assessment of Value depends on the stakeholder evaluating it, the specific medical condition and its treatment whose value is being evaluated. We define a stakeholder vector (Payer, Employer, Provider, Consumer (Patient)) or PEPC for this work. The proposed framework via Eq. (1) has the ability to accommodate this diversity, as it is based on integrating Value criteria from all 4 stakeholders by allowing the choice of different predictor attributes and different weights as well in the calculation of Q, C and V. This is achieved by partitioning both the Q and C factors of Eq. (1) as a common component (Q_{c} and C_{c}) common to all four stakeholders, and a specialized component (Qₛ and Cₛ) unique to a given stakeholder.

### B. Value and Scale

Kindig and Stoddart (2003) defined Population health as “the health outcomes of a group of individuals, including the distribution of such outcomes within the group.” We suggest that Individual and Population are two endpoints on a scale, and all four stakeholders need the assessment of Value at both endpoints. The value criteria in both cases are likely to be different depending on the stakeholder and scale. Each Patient undergoing a treatment would assess value in terms of Costs and Outcomes as an Individual, and yet Patients as a community would assess the value of treatment somewhat differently as a Population. Provider would assess a treatment's value in terms of not only the patient's well-being and cure, but also the quality, efficiency, risk and performance of their organization itself. Value criteria by stakeholder at both endpoints of the population scale need to be developed, which become the basis for predictor variables of C and Q.

### II. Value Calculations

The proposed analytic framework, based on Eq. (1), requires computation of Cost (C) and Quality Outcome (Q) metrics. Numerator and denominator of Eq. 1 respectively represent the algorithms for their quantification. They can be understood as multivariate expressions of Q and C in terms of several optionally advantageous predictor variables and weights, obtainable from measured data. Both numerator and denominator of Eq. 1 can hence expand into complex multivariate algebraic expressions with many variables, each capturing an aspect of outcome quality and cost respectively. Selection of these predictor variables and weights is a function of domain expert judgment. In addition, multivariate regression models can also be built using the same expressions for the prediction of C, Q and V. The predictor variables influencing this are specific to each disease condition, stakeholder considered (PEPC) and the scale (Individual or Population scale). In the below sections, we illustrate the proposed methods using Total Hip Replacement (THR) use case, both for the analysis and forward prediction purposes, separately.

In THR, diseased parts of the hip joint are replaced with new, artificial parts, called the prosthesis, which carries a risk of complications requiring revision surgery to correct the problem. Mahomed et al. (2003) reported that the rates of primary THR were three to six times higher than the rates of revision. Factors associated with an increased risk of an adverse outcome included increased age, gender (men were at higher risk than women), race (blacks were at higher risk than whites), a medical comorbidity, and a low income.

### A. Cost Factor C

Anders, et al (2013) assert intangible costs like transportation, out-of-pocket payments to the Provider, loss of wages, childcare due to illness as important. Regression methods proposed here capture such costs. Costs Cj per each the THR treatment episodes are collected from the actual costs incurred as per the patient record. An embodiment can use the Costs matrix shown in Table 2 for the THR Value calculations. Integration of costs in the last column of Table 2 can provide the denominator value in Eq. (1) for THR. This implementation is based on the premise that, an optimal Value possible is 1, when both the normalized numerator (Quality Q) and denominator (Cost C) are each equal to 1. This is not the best possible value for V, because there may be instances where the costs are so low and outcomes so excellent that the resulting Value (V) could be more than 1. The proposed algorithms are functional even in such rare cases, but it is reasonable to propose that the typical economic value V using the normalization of C and Q above would be in the range of 0 to 1 or at most <0 to 2>.

Normalization of Cost parameter C is achieved as follows:

A set of attributes contributing to Costs is selected

Cost predictor attributes are categorized into financial costs and intangible costs.

Weights wᵢ are assigned to the two predictor families such that their sum is always equal to 1

The financial (dollar) component of total cost C is normalized using an industry average $ cost value.

The intangible cost values also are normalized using an industry average metric on a Likert scale such as 1-10.

Contributions of each predictor are added as per Eq. 1 (numerator) and expressed as a %, the resulting C value would always be within the range {0, 1}.

In the above procedure, selection of the cost attributes, weights and intangible costs are finalized specific to each disease condition, in a process called Feature engineering in Machine Learning. This process of attribute and weight selection uses a combination of domain expertise about the disease and its treatment, and feature selection algorithms such as the Principal Components Analysis (PCA). This procedure is detailed for two different disease conditions, in this application in later sections.

B. Quality Factor Q

Quality of care is the degree to which health services for individuals and populations increase the likelihood of desired health outcomes and are consistent with current professional knowledge. According to Chung and Shauver (2009), Quality assessment maps to three domains: Structure, Process and Outcome. The Quality measures listed in Table 1 capture many of these three-category quality outcomes. The Patient Satisfaction attribute itself depends on multiple predictor variables. For example, a patient's ASA (American Society of Anesthesiologists) score is a subjective assessment of a patient's overall health that is based on five classes (I to V), I indicating a completely healthy fit patient to V indicating a moribund patient who is not expected to live 24 hour with or without surgery. Clearly, this score would have a significant impact on patient's satisfaction. Based on the cases of 712 THR recipient patients in Denmark, Husted et al. (2007) reported that age, sex, marital status, co-morbidity, preoperative use of walking aids, pre- and postoperative Hemoglobin levels, the need for blood transfusion, ASA score, and time between surgery and mobilization are the key variables which influence postoperative outcome in general, and patient satisfaction in particular. For the Outcome quality calculation for the THR use case, we use CMS Hospital Quality Measures (Table 1). Normalization of the Quality parameter Q is similar to that of Cost, discussed earlier. Weights wᵢ represent the relative importance, and can be assessed by experts using a modified wideband Delphi method. In the following sections, the key predictor attributes of THR outcomes Quality (Q) are discussed in detail.

HEDIS Scores: The Healthcare Effectiveness Data and Information Set (HEDIS) is a performance measurement tool that is coordinated and administered by NCQA (National Committee for Quality Assurance), structured as data collected on 71 measures over 8 domains to compare health plans and hospitals. It is one of the most widely used sets of health care performance measure in the U.S. HEDIS makes it possible to compare the performance of health plans on an “apples-to-apples” basis for a range of health issues, and is used by more than 90 percent of America's health plans to measure performance on important dimensions of care. HEDIS standards are a useful tool for comparing both the prevalence of chronic health problems, such as asthma, and the performance of health care delivery systems in responding to such problems. As a performance measure, HEDIS provides a set of technical specifications that define how to calculate a “rate” for some important indicator of quality. For instance, one HEDIS measure defines very precisely how plans should calculate the percentage of members who should have received beta blockers that actually were given a prescription. Using these measures, plans can determine what their rate is and how they compare to other plans. In Table 2, we include HEDIS metric for THR as a predictor variable contributing to the total Quality outcome (Q) for THR, along with a weight.

Harris scores for THR: The HHS was developed for the assessment of hip surgery, and is intended to evaluate various hip disabilities and methods of treatment in an adult population. The original version was published 1969. The HHS is a clinician-based outcome measure administered by a qualified health care professional, such as a physician or a physical therapist. It is a comprehensive framework to evaluate the quality of wellness after THR in terms of a patient's ability to walk, climb stairs, rotate etc. We have included HHS as a predictor variable (Table 1).

Physician Quality Reporting System score: PQRS enables individual Eligible Professionals (EPs) and group practices to report quality of care to Medicare. Quality Metrics were assessed (Qᵢ in Eq. 1) using the above THR outcome quality criteria, summarized in Table 1, along with the weights assigned to each attribute shown in [ ] as a % weight. These are all continuous (i.e., numerical) attributes. In contrast, some of the key Quality attributes can be categorical or dichotomous. For example, the probability of prognosis (i.e., treatments outcome) for THR is a 4 category attribute of states (PPM): Successful primary; Revision THR; Successful revision; Death, which we have included as a categorical variable in simulations reported below using Logistic Regression. In addition, the below outcome metrics also can serve as additional Quality determining attributes: Pain, Stiffness, Physical Function; QALY expectancy. The quality-adjusted life-year (QALY) is a measure of disease burden, including both the quality and quantity of life lived.

### III. Machine Learning for Value Prediction

Both Numerator (Q) and Denominator (C) of Value Eq. (1) contain 2 parts—a generic term applicable to all stakeholders and a specific term as explained earlier. Q and C each represent a Regression equation, obtained specific to each stakeholder's predictor attributes, using multivariate regression when predictors are all continuous (numerical) variables. Often, the predictor variables influencing Q and C are a mix of categorical, continuous and binary variables, and even unstructured data (clinical texts). In this case, one would use Logistic regression with Dummy coding for encoding of categorical variables. In Eq. 1, the aggregate Outcome Quality and Cost embodied in the Numerator and Denominator respectively are such predicted dependent variables, which in turn predict Value (Eq. 1). This is the basis of learning V based on newer data sets and features. Advantages of regression analysis approach is that it can be used to understand the relative importance of the predictor attributes to the output variables Q, C and V ultimately. A general linear regression problem can be explained by assuming some dependent or response variable y, (Cost, Quality outcome) which is influenced by inputs or independent variables xi₁, xi₂, . . . , xi_{q} (e.g. various contributions to cost or healthcare outcome). This relation can be expressed by a regression model [16]:

yi=β1+β2 xi2+ . . . +βq xiq+ε [2]

where β₁, β₂, . . . , β_{q} are fixed regression parameters and c is a random error or noise parameter. In an embodiment, a multiple linear regression via Eq. (2) is used to separately model the prediction of Cost (C) and Quality outcome (Q) for THR treatments. We then calculate Value V using Eq. (1).

In some cases, it would be more meaningful to assess whether a particular patient or population's care Value V is acceptable or not (i.e., High or Low satisfaction). Logistic regression is more suitable here, to analyze the relationship between a dichotomous dependent variable and one or more categorical or continuous independent variables. It specifies the likelihood of the response variable as a log of the odds ratio. Further, in case of both Cost and Outcome equations (2 and 3), it is possible to have predictor variables Xᵢ as dichotomous variables, such as Mortality status—whether alive (Y/N) or a multiple categorical variable, such as Degree of Pain (Acute, High, Moderate, Low, None). Dummy Coding is used to accommodate such non-numerical variables and yet complete a regression analysis on the mixed variable type dataset. It optionally advantageously involves coding the categorical predictor variables as 0 for Deceased and 1 for Alive; 0, 1, 2, 3 and 4 to encode the pain metrics Acute, High, Moderate, Low, None etc.

Application of any of the regression analyses described requires a training data set and a test data set, where the outcome variables (C, Q and V) are known a priori. Such datasets become feasible after the proposed framework is used in the industry for at least a limited time on a pilot basis. To overcome this difficulty, we prepared a synthetic data set for the THR use case comprised of 500 fictitious patients and used the same to train and test the regression models. Three such separate data sets are used for the GLM, Logit and Dummy Coding cases respectively. The predictor variables included in each case are summarized in Table 1 and 2. For the simulations, Institutional costs (INST), Personal costs (PERS) and Intangible (dissatisfaction/accessibility) costs (INTNG) as perceived by the Patient are also included. These are not shown in Table 1. Results from the 3 regression analyses, using the R package, are shown below.

### IV. Results & Discussion

Shown in Table 1 are the results from the Quality (Outcome) metric Q for THR. The stakeholder considered for this assessment is Patient at the scale of an Individual. A total of 8 predictor variables were selected based on THR domain. Weights wᵢ attributed to each of the 8 predictor variables of Quality (Outcome) metric Q for THR are shown within brackets [ ] in the first column of Table 1. For example, the THR Harris Score is assigned a weight of 70% whereas the fact whether Prophylactic Antibiotic was Received is assigned 2.5%. We have decided upon these weights using the Wideband Delphi method of weights assessment. These weights are subject to rigorous scrutiny before the industry would adopt the proposed framework.

TABLE 1
THR QUALITY (Q) CALCULATIONS EXAMPLE

| Quality (Q) Predictors | Measured (%) | Contribution |
|---|---|---|
| Hospital Surgery Metrics | | |
| PA Received 1 Hour Prior to | 60 | 1.5 |
| Surgical Incision [2.5%] | | |
| PA Selection for Surgical | 70 | 1.75 |
| Patients [2.5%] | | |
| PA Discontinued within 24 hrs. | 100 | 2.5 |
| After Surgery [2.5%] | | |
| Recommended VTE | 100 | 2.5 |
| Prophylaxis Ordered [2.5%] | | |
| THR Harris Score [70%] | 73 | 51.1 |
| HEDIS Score Metric [6%] | 78 | 4.68 |
| PQRS Measure [4%] | 67 | 2.68 |
| Patient satisfaction [10%] | 65 | 6.5 |
| Total Quality of Outcome | | 73.21 |
| Normalized outcome Q | | 0.7321 |

PA = Prophylactic Antibiotic;
VTE = Venous Thromboembolism

For example, the HEDIS metrics framework proposed earlier has received similar scrutiny before it is now widely adopted in the U. S. healthcare. The “Contribution” column (Table 1) shows the final contribution of the quality predictor to Q. Shown in Table 2 are the results from the Cost metric C for THR treatment use case. The stakeholder considered for thisassessment is Patient and the scale of an Individual. A total of 12 predictor variables were selected for this assessment based on research of earlier reports on THR. Weights wᵢ attributed to each of the 3 predictor variables of Cost metric are as follows: 70%, 20% and 10% respectively for the Institutional costs (INST), Personal costs (PERS) and Intangible (dissatisfaction/accessibility) costs (INTNG) as perceived by the Patient. Value calculation for THR (Table 3), using results from Tables 1 and 2 integrated via Eq. (1).

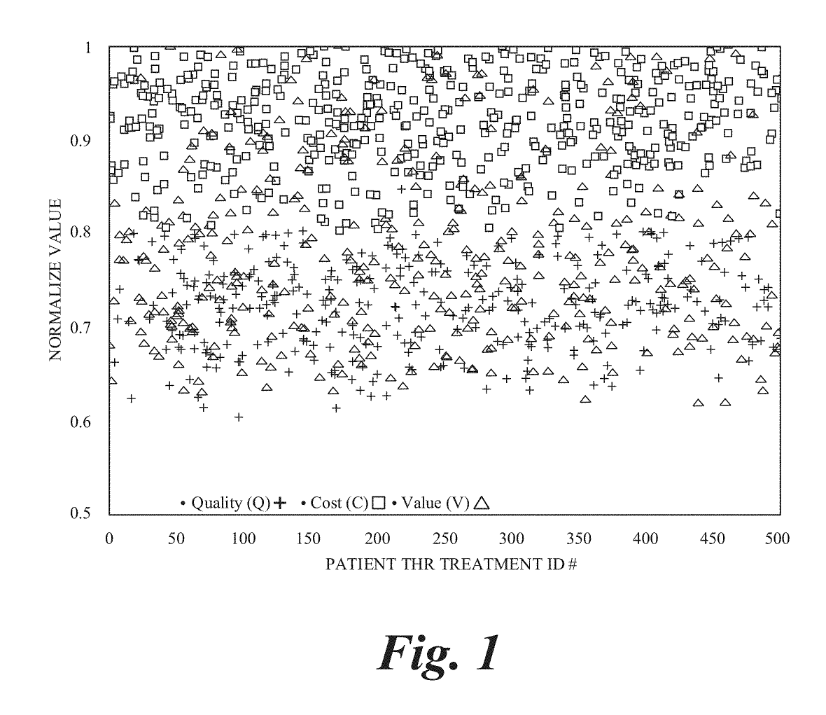

Shown in FIG. 1 are results from calculations of Q, C and V for a synthetic dataset comprised of the detailed predictor attribute data for both the Quality (Outcome) and Cost (C) calculations. It can be seen that the Value of THR treatment for these 500 cases varies between 0.5 and 1.1, 1 being an ideal value outcome. Value (V) rarely crosses a value of 0.9 in this simulation. A key factor determining the final normalized value is the national average cost of THR treatment, which is reported to be $30,124. Since the Institutional cost of the treatments in the current data set for majority of patient cases are close to this value, as per Eq. 1, V values are closer to 1, the ideal value (FIG. 1). The ML algorithms predict these coefficients β₁, β₂, . . . , β_{q}, as a function of the actual assessed Value as reported by the individual patient instances which form the training set for regression.

TABLE 2
THR cost (C) calculations example

| Cost Item | Symbol | Cost ($) |
|---|---|---|
| Room & Bed General Class | [ROOM] | 3,050 |
| Pharmacy | [PHARMA] | 586 |
| Medical/Surgical supplies | [SURGCL] | 684 |
| Non Sterile supply | [NONSTR] | 117 |
| Sterile supply | [STERL] | 1,366 |
| Supply/IMPLANTS | [IMPNT] | 9,315 |
| Laboratory | [LAB] | 1159 |
| Radiology/Diagnostic | [RAD] | 322 |
| OR Services | [ORS] | 5,491 |
| Anesthesia | [ANSTH] | 855 |
| Physical Therapy | [PHYSIO] | 552 |
| PT eval/Re-eval | [PT] | 347 |
| Occupational Therapy | [OCT] | 240 |
| Required Drugs | [DRGS] | 4058 |
| Self-administered Drugs | [SELF] | 400 |
| Recovery Room | [REC] | 804 |
| EKG/ECG | [EKG] | 125 |
| Peripheral Vascular Lab | [VASC] | 310 |
| Total Cost | | 29,781 |
| National Average Cost | | 30,124 |
| Normalized Cost C | | 0.93 |

Shown in Tables 3 and 4 are results from multiple linear regression separately to predict Q and C, using R software, Machine Learning implementation software, applied to the synthetic 500 patient dataset. In Run 1 and Run 2, Multiple Linear regression (GLM) is used to assess Q and C; and in Run #2, Logistic Regression is used to assess Q and Dummy Coding is used to encode the Probability of Prognosis Metric (PPM) as a categorical variable as per the standard industry practice: S=successful primary (0); R=Revision THR (1); SR=successful revision (2); D=Death (3). Cost C prediction model (GLM) is shown in Table 4. While these results do not necessarily reflect the goodness of fit using data measured in clinical settings, they illustrate how

Value for VBC can be predicted using the selected predictor attributes of Quality outcomes (Q) and Costs (C), which is novel and would be significantly helpful to the VBC practice. McFadden's R² statistic indicates the proportion of variance in the dependent variable, explained by the predictors in a Logistic regression model. Its low value for Run #2 indicates that the Logit model fit has a low accuracy. FIG. 1 illustrates Q, C and V calculations for 500 THR patients.

The intent of this simulation is mainly to demonstrate the feasibility of conducting regression on the Value data sets even when some of the outcomes being predicted (Q or Quality outcome in this case) are dichotomous or categorical. Hence, rigorous goodness of fit analyses for the results from the regression model fitting are not detailed in this work. This is the focus of our ongoing modeling efforts.

TABLE 3
REGRESSION COEFFICIENTS: GLM AND LOGIT ON Q

| | |
|---|---|
| Run #1 | Run #2 |

| | Estimate | Std. Err. | Estimate | Std. Err. |
|---|---|---|---|---|
| PAR | 0.1891 | 0.0380 | 0.0009 | 0.0005 |
| PAS | 0.1270 | 0.0392 | 0.0014 | 0.0006 |
| PAD | 0.1040 | 0.0376 | 0.0018 | 0.0005 |
| VTE | 0.1778 | 0.0389 | 0.0018 | 0.0006 |
| HSS | 0.1306 | 0.0378 | 0.0014 | 0.0005 |
| HEDIS | 0.1302 | 0.0380 | 0.0011 | 0.0005 |
| PQRS | 0.0955 | 0.0377 | 0.0013 | 0.0005 |
| PSAT | −0.0117 | 0.0091 | 0.0004 | 0.0001 |
| PPM | | | −0.0013 | 0.0032 |

### Advantages of the Framework

A fully quantitative analytic framework is presented for the assessment of Value in Value Based Care, which is a critical capability required by the Healthcare industry. No comparable methods are available in the academic or industrial practice so far. It is capable of assessing Quality, Cost and Value specific to any of the many different disease conditions, treatments and outcomes, taking into account the various key predictors (features) of such conditions, treatments and outcomes. Further it fits seamlessly into the modern big data analytic frameworks via several Machine Learning algorithms such as Neural Networks, Decision Trees and Support Vector Machines among others for example, to achieve classification of treatments by Value. Influence of categorical variables such as patient satisfaction, pain and morbidity levels, as well as semi-structured and unstructured data such as clinical transcripts can be factored in via Machine Learning methods, by extracting the relevant information from unstructured data sources as features (attributes) and adding them to the datasets used in the calculation or prediction of Value. Furthermore, features influencing Q, C and V can be engineered using methods such as Principal Components Analysis (PCA) and Linear Discriminants Analysis (LDA).

TABLE 4
REGRESSION COEFFICIENTS: GLM ON COST C

| Predictor | Estimate | Std. Error |
|---|---|---|
| ROOM | 3.53E−05 | 1.23E−05 |
| PHARMA | 1.56E−05 | 4.18E−05 |
| SURGCL | 7.93E−05 | 4.11E−05 |
| NONSTR | 9.63E−05 | 1.30E−04 |
| STERL | 5.16E−05 | 2.50E−05 |
| IMPNT | 7.38E−06 | 3.99E−06 |
| LAB | 3.71E−05 | 3.12E−05 |
| RAD | 8.40E−05 | 6.48E−05 |
| ORS | 2.00E−05 | 5.04E−06 |
| ANSTH | 1.39E−04 | 4.13E−05 |
| PHYSIO | 7.04E−05 | 4.23E−05 |
| PT | 2.19E−04 | 8.10E−05 |
| OCT | 1.11E−05 | 6.29E−06 |
| DRGS | 6.19E−05 | 4.29E−05 |
| SELF | 2.82E−06 | 3.17E−05 |
| REC | 2.34E−04 | 9.10E−05 |
| EKG | 1.59E−04 | 6.35E−05 |
| VASC | 1.29E−04 | 6.35E−05 |
| PERS | 3.43E−06 | 2.52E−06 |
| INTNG | 1.99E−04 | 1.28E−04 |

An additional advantage of ML based methods presented is their predictive power, which analysts could use to learn the Value of various healthcare options at an individual as well as population scale. The framework can be adapted to specific stakeholders' needs and viewpoints to plan, track, assess and agree upon the Value of healthcare services. While the present work demonstrates an internally consistent Value assessment framework, there are several gaps which must be addressed before the framework can be widely used in healthcare. Eqn. 1 actually represents a fundamental algorithm for the assessment of V, Q and C, which each would expand into thousands of specific cases of calculation, depending on the medical condition, treatment and stakeholder, each in turn influenced by dozens of predictor variables, specific to each case. For example, the Harris Score (HSS) metric used in this study is specific to THR, whereas there are very different predictors for other conditions such as the coronary disease etc. The proposed methods need to be tested on such different disease and treatment conditions. A more rigorous, comprehensive evaluation of the predictor variables and their weights is needed to establish the accuracy of the proposed methods. Similarly, collection of practical, independent datasets for training and testing the proposed models is a critical need. Despite these limitations, the proposed analytical framework represents a first fully quantitative, comprehensive analytical and learning tool for the assessment of Value in VBC.

### Detailed Method Description Applied to COPD

Here, we report on its application to assessing the Value of treatment of Chronic Obstructive Pulmonary Disease (COPD)—third leading cause of death in the U.S., costing $ 50 billion annually and affecting 24 million people. Assessment of quantitative value depends on the stakeholder evaluating it and the specific medical condition and its treatment whose value is being evaluated, along a stakeholder vector (Payer, Employer, Provider, Consumer (Patient) or PEPC. Value of treatment for a specific stakeholder (PEPC) is defined as [Eq. 1] where Qᵢ represent the health quality measures carrying Weights w₁, which contribute to the net quality of care Q. Similarly, represent the costs of delivering the care, while eⱼ is the episode associated with a particular cost contribution Cj. For example, surgery, office visits, and pharmacy are 3 different costs within a care episode which all contribute to the total cost C. In addition, non-monetary costs, such as pain and difficulty of access to treatment, which typically are expressed as categorical variables on a Likert scale (as opposed to $ costs) can also be included in the cost factor C. Expressions for Q and C in Eq. (1) can not only compute value when all cost and quality metrics and their relative importance via weights are known a priori, but also classify and predict Q, C and V via ML algorithms when the set of direct cost and quality predictors and their relative influence are unknown. Further, the proposed framework is able to accommodate the diversity of stakeholders, by integrating value assessment criteria from all 4 stakeholders, via selection of different predictor variables (attributes) and different weights as well in the calculation of Q, C and V. Present work limits its focus to Patient (P) as a stakeholder at the scale of an Individual, for COPD disease.

### V. COPD Outcomes

Glaab et al (2012) reported outcome measures commonly applied in current COPD trials. We selected the below outcome measures to assess Q as via multi-variate linear regression among these weighted attributes.

Lung Function: is characterized by FEV1/FVC (volume of air that can forcibly be blown out after full inspiration, and the volume of air that can forcibly be blown out in one second, after full inspiration, respectively) and IC/TLC Ratio (Total Lung Capacity to Inspiratory Capacity ratio). FEV1/FVC is about 70-85% in healthy adults, declining with age, and as low as 45% in case of COPD, because of increased airway resistance to expiratory flow. We used the FEV1/FVC ratio and specify its range as 40 to 85%.

Exercise Capacity Metrics: are a key measure of COPD patients' overall function, which include 6-Minute Walk Test (6MWT), Shuttle Walk Test (SWT) and Ergometry Test Score. We use the 6-Minute Walk Test (6MWT).

Dyspnea Measures: include BDI/TDI [Baseline Dyspnea Index/Transition Dyspnea Index (BDI/TDI)], Borg Scale (CR-10) and Medical Research Council (MRC) scale, as measures of dyspnea (a subjective sensation of difficulty in breathing), which itself is an effective measure of COPD treatment outcomes. In the BDI each of the three categories (functional impairment, magnitude of task and magnitude of effort) that are useful for quantifying the limitation due to dyspnea) has 5 levels of symptom severity from 0 to 4 where 0 corresponds to the most severe level. BORG scale is used to assess shortness of breath on a scale of 0 to 10, 0 implying no shortness of breath and 10 an extreme shortness of breath. MRC score's 0 represents the lowest level of dyspnea and level 4 the greatest impairment.

Health Status Metrics: also are crucial to evaluating COPD treatment effectiveness, which can be characterized by the St. George's Respiratory Questionnaire (SGRQ), Chronic Respiratory Disease Questionnaire (CRQ) and Medical Outcomes Study Short Form-36 (SF-36). Among these, CRQ is an interviewer-administered questionnaire measuring both physical and emotional aspects of COPD, across four dimensions: dyspnea, fatigue, emotional function, and mastery. On its 0-7 scoring scale, lower score indicates better health; for example 1=“not tired at all”, 7=“extremely tired.”

Exacerbations Endpoints are “characterized by a change in the patient's baseline dyspnea, cough and sputum that is beyond normal day-to-day variations, is acute in onset and warrants a change in regular medication”. We include Frequency of exacerbations, Time to first exacerbation, Severity and Duration for assessing Q, each with an equal weight of 2.5% (Table 1). These metrics map to a 0-10 Likert scale, 0 being least severe impact.

BODE Score, a multidimensional system widely used to assess COPD treatment, comprises four components—nutritional state (BMI), airflow limitation (Obstruction; FEV1), breathlessness, (MRC Dyspnea scale) and Exercise capacity (6MWD, distance walked in 6 min). It is given a high weightage (50%) here, as it is a well-accepted and comprehensive measure. On a 0-10 scale, higher BODE scores correlate with higher risk of death.

Shown in Table 1 are the final COPD quality measures selected in this study (Column 1 of Table I), with their relative weights (wᵢ) and ranges shown in Column 2. All of the quality metrics selected indicate a worsening quality with increasing value, excepting the BODE score, MRC and BDI/TDI, which indicate a worse quality at a lower value (indicated by * in Table 1). Their contribution to the overall degradation of quality (Column 4) is assessed by subtracting their value from the maximum value of their range and using the result for the final contribution (Column 4). Summing over the normalized contribution values against all quality metrics results in a final degree of degradation in Q; this value subtracted from 100 is the Quality (Q) measure in Eq. (1). Using synthetic measured values for COPD quality in Table 1 (Column 3), we quantify the final Quality metric Q for the particular patient as 5.9. Automating this process over a synthetic data set of 160 patients, we assessed their Q, C and hence V metrics detailed next (Table 1 and 2).

### VI. COPD Cost Attributes

Global Initiative for Chronic Obstructive Lung Disease (GOLD) divides worsening of COPD into 4 stages. Hospitalization costs are high in stage 4 and low in stage 1.

TABLE I
COPD QUALITY METRICS AND QUALITY (Q) CALCULATION

| Quality | Range* | Measu | Cont |
|---|---|---|---|
| FEV1/FVC | [10%, 40- | 60 | 0.55 |
| 6-Minute Walk | [10%, | 6 | 0.60 |
| BDI/TDI | [4%, | 3 | 0.10 |
| Borg Scale | [3%, | 8 | 0.24 |
| MRC | [3%, 0..4] | 2 | 0.15 |
| CRQ | [10%, | 5 | 0.71 |
| BODE Score | [50%, | 8 | 0.72 |
| Exacerbations | [2.5%, | 7 | 0.17 |
| 1st exacerbation | [2.5%, | 6 | 0.15 |
| Severity | [2.5%, | 8 | 0.20 |
| Duration | [2.5%, | 7 | 0.17 |

^{a}Lower value means worse outcome

Dalal et al (2010) assessed Direct costs of COPD among 37,089 managed care patients incurred at various sites of care, and reported that 53%, 37%, 3% were in the outpatient, urgent outpatient and ED cohorts respectively, and the standard admission and ICU cohorts together comprised 6%. Mean annual COPD-related health care costs increased across the cohorts, ranging from $2,003 to $43,461 per patient. They obtained data for this retrospective analysis from a managed care claims database which included geographically diverse commercial health plan members in the US, for approximately 14 million commercial enrollees. We use this study, for example, as a basis for cost metrics.

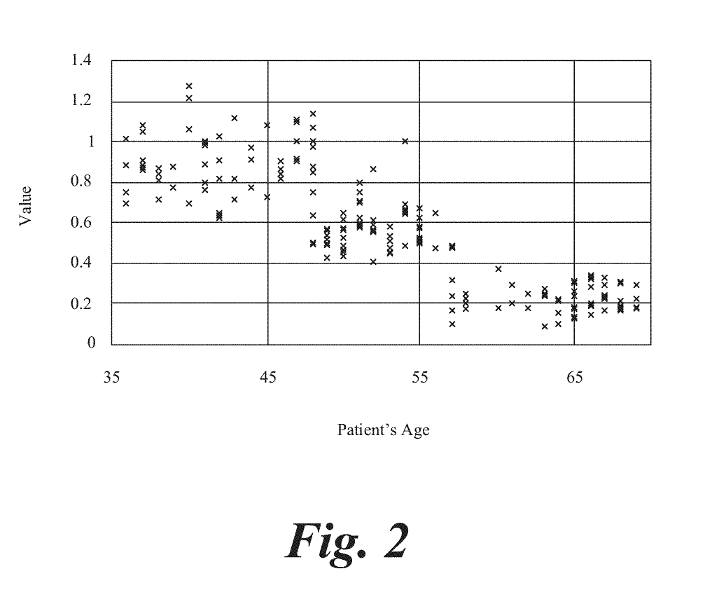

Dalal grouped the patients into five mutually exclusive cohorts—Outpatient cohort who had at least one medical claim for office, lab or outpatient care with COPD, “urgent outpatient” cohort who had at least one medical claim for outpatient care for COPD followed by a pharmacy claim, ED cohort had at least one medical claim for an ED visit for COPD but no evidence of an inpatient stay for COPD, “standard admission” cohort comprised patients with at least one inpatient stay for COPD, and “ICU” cohort had evidence of ICU care during a COPD inpatient stay. Cost metrics map to pharmaceutical costs and hospital treatment costs. FIG. 2 illustrates Value (V) as a function of patient age for a 150-patient dataset.

We combined these costs into a single line item (Table 2). Depending on which of the 4 stages of COPD treatment a patient is in, these costs would be different. Following the cost assessment by Dalal et al we used the median costs shown in Table 2 for C values in Eq. (1). Depending on the COPD stage, treatment cost and value would be different. The example calculation of Q for a single patient shown following Table 1 corresponds to stage 2. Accordingly, using the corresponding normalized cost of $4,000, Value of COPD treatment for this patient would be 0.69, against a maximum of 1.0 to 1.2. While the ideal value of V would be 1, high quality outcomes at subsidized costs can indeed result in a high value number such as 1.2 or greater.

Using the synthetic data set, Q and C values for a set of 160 patients were calculated, and their COPD treatment value (V) was assessed via Eq. 1 (results shown in FIG. 1). Trends in V as a function of patient's age show a step-wise degradation in V with patient's age. The data set was prepared such that the key outcome metrics (Table 1) worsen with age, and the treatment costs also increase with age and maps to advanced (worse) stages of COPD, which is a reasonable assumption. COPD is a chronic condition to be managed not cured, and as a patient ages, they develop additional comorbidities which have interactive effects with COPD. Results shown in FIG. 1 reflect this, as 3 distinct “value bands”—the average V values being 0.90, 0.58, and 0.23 respectively for the 3 age bands (steps seen in FIG. 1). While this analysis is based not on real clinical data, the synthetic data based analysis of Value V clearly demonstrates the usefulness of the proposed methodology. For example, COPD population dynamics including the expected quality (Q), costs (C) and Value (V) of treatments can be assessed.

For the above, we constructed a 25 attributes dataset for 160 patients, with 11 Outcome (Q) attributes (Table 1), 10 cost attributes (Table 2), and the following 4 risk characterization attributes: Age, Smoking status, BMI and Stage of COPD (0 to 3). This synthetic dataset is intended to illustrate the development and application of ML for the classification of V, Q and C, and should not be construed as an accurate reflection of the disease status among a population. These values are selected from a plausible range appropriate for the given patient risk profile, but does not represent a truly measured clinical dataset. For example, a high cost and low quality outcome patient instance would correspond to a combination of advanced age, high BMI (>30), long smoking history and advanced (severe) COPD stage, for example. We extend this method for classification of the same set of patients by Value, when only COPD risk factors are known (Q and C unknown), via ML methods as below.

TABLE II
COPD COST METRICS AND COST (C) CALCULATION

| | Cost by Cohort/Stage (USD) | | | | |
|---|---|---|---|---|---|
| Institutional | | Urgent | | Stad | |
| Cost Item | OutP | OutP | ED | InP | ICU |
| Pharmaceutical | 999 | 1423 | 14 | 1533 | 1734 |
| Room and Bed | 0 | 0 | 18 | 7200 | 16200 |
| | 600 | 900 | 90 | 3000 | 11400 |
| ED charges | 200 | 300 | 30 | 1000 | 3800 |
| ICU Charges | 117 | 175.5 | 17 | 585 | 2223 |
| Physician Fees | 140 | 210 | 21 | 700 | 2660 |
| Staff Fees | 70 | 105 | 10 | 350 | 1330 |
| Laboratory | 94 | 141 | 14 | 470 | 1786 |
| Recovery | 55 | 82.5 | 82. | 275 | 1045 |
| EKG/ECG | 45 | 67.5 | 67. | 225 | 855 |
| Total Hospital | 132 | 1981.5 | 37 | 1380 | 41299 |

### Classification for Value

The main cause of COPD in developed countries is tobacco smoking. In the developing world, COPD often occurs in people exposed to fumes from burning fuel for cooking and heating in poorly ventilated homes. Only about 25 percent of chronic smokers develop clinically apparent COPD, although up to half have subtle evidence of COPD. About 85 to 90 percent of all COPD cases are caused by cigarette smoking. Obesity, defined as corresponding to a BMI greater than 30 kg/m², also has severe compounding effects on COPD. Age also has a key influence on COPD, which occurs most often in older adults, affects people in their middle ages but not common in younger adults. Further, it takes several years for COPD to develop. Therefore, we suggest that smoking status (smoker Heavy/moderate/None), Age (<40, 40-55, >55) and BMI (<25, 25-30, >30) as effective influencing attributes on the Quality of treatment effectiveness Q as well as the Costs incurred for treatment C. That is, a patient at high risk for COPD due to a combination of the above three factors would have high cost of treatment and lower quality outcomes, thus leading to a low Value V. This is only a plausible hypothesis, not validated in the present study. However, it enables preparation of a synthetic dataset (Table 3) to illustrate how ML can be used to predict Value V of COPD treatment for a given patient, given their risk factor data. For example, these variables can be used to adjust for acuity (risk adjustment), a common practice when evaluating healthcare.

Two additional datasets were prepared from this synthetic dataset, as training data and test datasets used to classify the data based on Value V, as a function of only four key risk measures: smoking status, age, BMI and stage of COPD progression. A part of this dataset is shown in Table 3. In medical analytics, datasets with such risk metrics are relatively easier to obtain, whereas datasets with actual treatment costs and outcomes for patients (as in Tables 1 and 2) are very difficult to obtain, which is a major impediment to analytics. The ML based methods presented using indirect measures such as age help overcome this difficulty. Patient ID shown (Table 3) are spurious, and V values drawn from prior analysis as above.

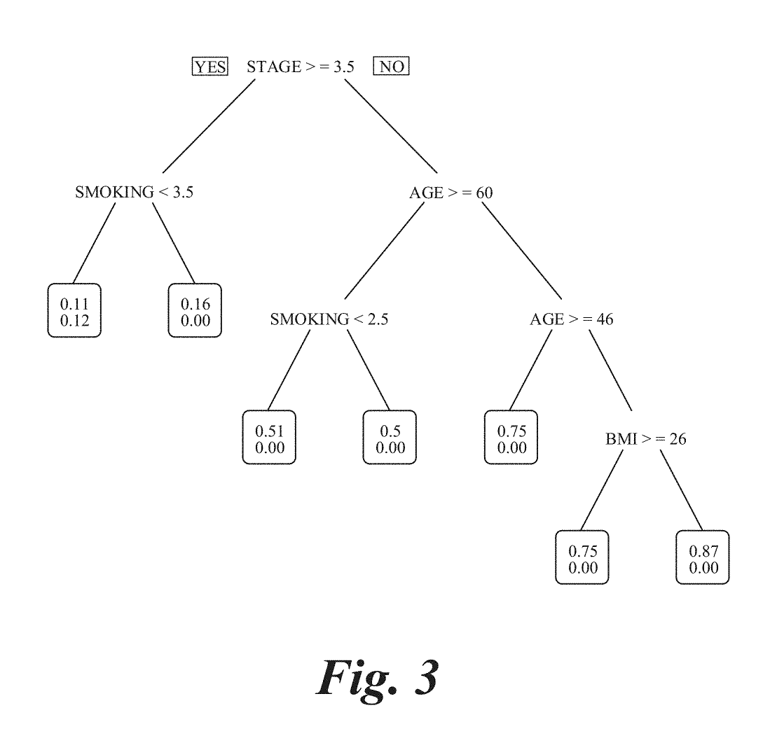

Using R ML package RPART on this type of dataset (Table 3), a decision tree for classification of COPD treatments by Value (FIG. 3) is constructed, which clearly illustrates that advanced stage COPD patients who are heavy smokers show least value of treatment (V around 0.11), whereas patients in early stages of COPD and who do not smoke show the highest value V. While these results are plausible, they do not represent conclusions based on actually measured data at all. They are intended only to illustrate how the novel value analytic platform according to an embodiment can be used to assess value of COPD treatments based solely on patient risk measures, without any measurements of cost or treatment outcomes per se, and do not reflect actual COPD risk data.

TABLE III
COPD RISK FACTORS AND VALUE DATASET

| PATIENT | | | | | |
|---|---|---|---|---|---|
| ID | AGE | SMOKING | BMI | STAGE | V |
| BRC107 | 69 | 4 | 36 | 4 | 0.066871 |
| AMQ114 | 53 | 2 | 30 | 3 | 0.873197 |
| IWC009 | 46 | 2 | 24 | 2 | 0.792542 |
| KLX1084 | 37 | 2 | 23 | 2 | 0.76001 |
| BRC117 | 38 | 1 | 22 | 2 | 1.082441 |
| AMQ231 | 60 | 3 | 33 | 4 | 0.115766 |
| WSC3212 | 38 | 2 | 22 | 2 | 0.868661 |

The decision tree shown, with a RSME and AME values (measures of accuracy of prediction) of 0.93 and 0.68 respectively, indicates good accuracy of prediction. While these results are plausible, they are intended only to illustrate how the new quantitative value analytic platform can be used to assess value of COPD treatments based solely on patient risk measures, without any measurements of cost or treatment outcomes per se.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

What is claimed is:

1. A computer-implemented method, comprising the steps of:
defining a set of multiple stakeholder entities;
for each stakeholder entity of the set of stakeholder entities, defining a corresponding set of multiple health outcomes (Qᵢ);
defining a set of multiple costs (Cⱼ), each cost of the set of costs corresponding to a respective health outcome (Qᵢ) of the set of health outcomes; for each stakeholder entity of the set of stakeholder entities, determining a value (V) according to the equation
$$V = \frac{{\sum }_{i}^{n} {w}_{i} {Q}_{i}}{{\sum }_{j}^{m} {e}_{j} {C}_{j}} ,$$
wherein wᵢ represents a set of numerical weights and eⱼ represents a set of episodes associated with a particular cost (Cⱼ) to create a set of values (V); and
determining from the set of values (V) a numerical value (V′) optimal to the set of stakeholder entities.

2. The method of claim 1, further comprising the steps of identifying and evaluating variables that influence health outcome (Qᵢ), costs (Cⱼ) and values (V) based on machine learning (ML) algorithms.

3. The method of claim 1, further comprising the step of rigorously assessing Value (V) for treatments that occured in the past specific to any of multiple known diseases conditions.

4. The method of claim 1, further comprising the step of rigorously predicting Value (V) for treatments that are yet to be performed in future specific to any of multiple known disease conditions.

5. The method of claim 1, further comprising the step of rigorously prescribing one or more preferred treatment options by comparatively assessing the Value (V) of such treatments yet to be performed in future specific to any of multiple known diseases conditions using ML Recommender System algorithms.
